(19) United States
(12) Patent Application Publication
(10) Pub. No.: US 2017/0114367 A1
(43) Pub. Date: Apr. 27, 2017

(54) METHOD FOR BACTERIAL GENOME EDITING

(71) Applicants: NATIONAL TSING HUA UNIVERSITY, HSINCHU CITY (TW); CHANG CHUN PLASTICS CO., LTD, TAIPEI (TW); CHANG CHUN PETROCHEMICAL CO., LTD, TAIPEI (TW)

(72) Inventors: YU CHEN HU, HSINCHU CITY (TW); MU-EN CHUNG, HSINCHU CITY (TW); I-HSIN YEH, YUNLIN COUNTY (TW); HUNG LI, HSINCHU CITY (TW)

(21) Appl. No.: 15/066,063

(22) Filed: Mar. 10, 2016

(30) Foreign Application Priority Data
Oct. 26, 2015 (TW) 104135073

(51) Int. Cl. C12N 15/90; C12N 15/74; C12N 15/72

(52) U.S. Cl. CPC C12N 15/902; C12N 15/72; C12N 15/74

(57) ABSTRACT

A method for bacterial genome editing includes: providing a bacterial cell; transforming a pCas9 plasmid and a pKD46 plasmid into the bacterial cell; co-transforming a pCRISPR::LacZ plasmid and an exogenous DNA into the bacterial cell carrying the pCas9 plasmid and the pKD46 plasmid, so as to obtain a strain broth; and spreading the strain broth on a culture medium to conduct cultivation.

### BACKGROUND OF THE INVENTION

1. Field of the Invention

The instant disclosure relates to a method for bacterial genome editing; in particular, to a method for bacterial genome editing of using a CRISPR/Cas9 technique to increase genome editing efficiency of Escherichia coli and cyanobacteria.

2. Description of Related Art

When using a traditional genome editing technique to modify a bacterium for conducting metabolic engineering, some genes of the bacterium need to be knocked out or several genes need to be inserted simultaneously. If the several genes are inserted into the bacterium at the same time, a fragment composed of the several genes would be very big. Thus, the several genes need to be separately inserted into the bacterium several times. During the traditional genome editing process, antibiotics are added in a culture medium, the survival bacterium is selected, and the antibiotics are then removed. The above steps are repeatedly until the several genes are completely inserted into the bacterium, and the steps are complicated and time consuming.

CRISPR/Cas9 is a recent highly focused genome editing technique. Compared to the traditional genome editing technique, CRISPR/Cas9 can be used to knockout or insert the several genes at the same time, and genome editing technique of the CRISPR/Cas9 is relatively easier than the traditional genome editing technique, so as to increase convenience of genome editing. However, in past studies, the CRISPR/Cas9 is mostly used in mammalian genome editing, and it is rarely used in bacterial genome editing. Even if there are some research results about application of the CRISPR/Cas9 being used in bacterial genome editing, only small sizes of DNA fragments can be used, and the available DNA fragments are not larger than 3 kb. Otherwise, the efficiency of genome editing would be reduced.

In order to overcome the abovementioned drawbacks, the instant disclosure provides a method the CRISPR/Cas9 technique is mostly used in mammals, and it is rarely used in bacteria. Even though there are some studies of the CRISPR/Cas9 technique being used in bacteria, but only small DNA fragments being available to be inserted in the bacterium. When the DNA fragment as large as 3 kb, the efficiency of genome editing will be decreased.

Therefore, how to use the CRISPR/Cas9 technique to improve the efficiency of genome editing in the bacterium and overcome the abovementioned problems that is one of the important issues in the industry.

### SUMMARY OF THE INVENTION

In order to overcome the abovementioned problems, this instant disclosure provides a method for bacterial genome editing which can successfully insert large DNA fragments into a bacterium, and can increase a speed of genome editing in the bacterium, so as to improve a success rate of genetic recombination in the bacterium.

Accordingly, an embodiment of this instant disclosure provides a method for bacterial genome editing which includes following steps: providing a bacterial cell; transforming a pCas9 plasmid and a pKD46 plasmid into the bacterial cell; co-transforming a pCRISPR::LacZ plasmid and an exogenous DNA into the bacterial cell carrying the pCas9 plasmid and the pKD46 plasmid, so as to obtain a strain broth; and spreading the strain broth on a culture medium to conduct cultivation.

Preferably, in the method for bacterial genome editing of the embodiment in this instant disclosure, wherein after the steps of co-transforming the pCRISPR::LacZ plasmid and the exogenous DNA into the bacterial cell carrying the pCas9 plasmid and the pKD46 plasmid, further includes the following steps: using a Cas9 protein and a guide RNA which can identify a LacZ gene to specifically cut at a specific cutting site of the LacZ gene in the bacterial cell; and inserting the exogenous DNA into the specific cutting site of the LacZ gene in the bacterial cell, and obtaining the strain broth.

Preferably, in the method for bacterial genome editing of the embodiment in this instant disclosure, the bacterial cell is an Escherichia coli.

Preferably, in the method for bacterial genome editing of the embodiment in this instant disclosure, the culture medium contains isopropyl β-D-1-thiogalactopyrano side, 5-bromo-4-chloro-3-indolyl-β-D-galactopyranoside, Kanamycin, and Tetracycline.

Another embodiment of this instant disclosure provides a method for bacterial genome editing which includes the following steps: providing a bacterial cell; co-transforming a pHR_trc template plasmid and a pCas9-NSI plasmid into the bacterial cell to obtain a strain broth; and spreading the strain broth on a culture medium to conduct cultivation.

Preferably, in the method for bacterial genome editing of the embodiment in this instant disclosure, the bacterial cell is a cyanobacteria.

Preferably, in the method for bacterial genome editing of the embodiment in this instant disclosure, the pHR_trc template plasmid is used as a homologous recombination template, and contains a Spectinomycin resistance gene Spec^{R}, a fluorescent protein EGFP, and a homologous recombination region NSIa and NSIb.

Preferably, in the method for bacterial genome editing of the embodiment in this instant disclosure, the predetermined position is a double-strand break locus.

Preferably, in the method for bacterial genome editing of the embodiment in this instant disclosure, the culture medium is a BG-11 culture medium contains Spectinomycin.

The beneficial effects of the instant disclosure are, by the method for bacterial genome editing of the embodiment in this instant disclosure, large DNA fragments can be successfully inserted into a bacterium, and genetic recombination in the bacterium can be successfully conducted. Therefore, the method for bacterial genome editing of the embodiment in this instant disclosure can be used for increasing a convenience of genome editing in the bacterium, and can speed up the genome editing in the bacterium, so as to modify a bacterial DNA. In the future, the method for bacterial genome editing of the embodiment in this instant disclosure can be used for regulating a bacterial metabolic pathway to achieve a purpose of producing bio-chemicals, so as to replace a conventional oil cracking which entails a heavy pollution process.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 shows a flow chart of a method for bacterial genome editing of a first embodiment in the instant disclosure;

FIG. 2 shows a schematic view of various lengths of exogenous DNA of a first embodiment in the instant disclosure;

FIG. 3 shows a schematic view of a method for bacterial genome editing of a first embodiment in the instant disclosure; and

FIG. 4 is a blue/white screening result showing the number of colonies in Experiment 1 of a first embodiment in the instant disclosure;

FIG. 5 is a bar chart of blue/white screening result showing the number of colonies in Experiment 1 of a first embodiment in the instant disclosure;

FIG. 6 is a result of polymerase chain reaction based colonies rapid test in Experiment 1 of a first embodiment in the instant disclosure;

FIG. 7 is an analysis result of an exogenous DNA inserting into a chromosome in Experiment 1 of a first embodiment in the instant disclosure;

FIG. 8 shows a flow chart of a method for bacterial genome editing of a second embodiment in the instant disclosure;

FIG. 9 shows a schematic view of a method for bacterial genome editing of a second embodiment in the instant disclosure;

FIG. 10 shows a schematic view of a template plasmid in Experiment 2 of a second embodiment in the instant disclosure;

FIG. 11 is a result of homologous recombination efficiency of a cyanobacteria in Experiment 2 of a second embodiment in the instant disclosure; and

FIG. 12 is a bar chart showing the homologous recombination efficiency in Experiment 2 of a second embodiment in the instant disclosure.

### DETAILED DESCRIPTION OF THE PREFERRED EMBODIMENTS

Embodiments disclosed in the instant disclosure are illustrated via specific examples as follows, and people familiar in the art may easily understand the advantages and efficacies of the instant disclosure by the disclosure of the specification. The instant disclosure may be implemented or applied by other different specific examples, and each of the details in the specification may be applied based on different views and may be modified and changed under the existence of the spirit of the instant disclosure. The figures in the instant disclosure are only for brief description, but they are not depicted according to actual size and do not reflect the actual size of the relevant structure. The following embodiments further illustrate related technologies of the instant disclosure in detail, but the scope of the instant disclosure is not limited herein.

### First Embodiment

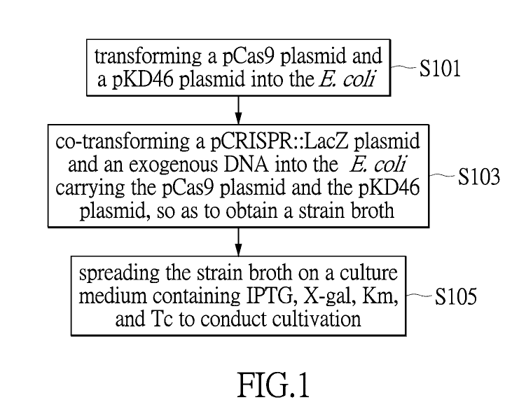

Please refer to FIG. 1. FIG. 1 shows a flow chart of a method for bacterial genome editing of a first embodiment in the instant disclosure, and S101, S103, and S105 represent steps of the method for bacterial genome editing. A first embodiment of this instant disclosure provides the method for bacterial genome editing, and a bacterial cell of the first embodiment of this instant disclosure is an Escherichia coli (hereinafter called as E. coli).

The first embodiment of this instant disclosure provides the method for bacterial genome editing which includes following steps: providing a bacterial cell; transforming a pCas9 plasmid and a pKD46 plasmid into the bacterial cell using an electroporation method; co-transforming a pCRISPR::LacZ plasmid and an exogenous DNA into the bacterial cell carrying the pCas9 plasmid and the pKD46 plasmid using the electroporation method; using a Cas9 protein (expression from the pCas9 plasmid) and a guide RNA (expression from the pCRISPR::LacZ plasmid) which can identify a LacZ gene to specifically cut at a specific cutting site of the LacZ gene in the bacterial cell; at this time, inserting the exogenous DNA into the specific cutting site of the LacZ gene in the bacterial cell, conducting a genetic recombination process, and obtaining a strain broth; and spreading the strain broth on a culture medium to conduct cultivation at 37° C. for 16 to 24 hours. Wherein, the culture medium contains isopropyl β-D-1-thiogalactopyranoside (IPTG), 5-bromo-4-chloro-3-indolyl-β-D-galactopyranoside (X-gal or BCIG), Kanamycin (Km), and Tetracycline (Tc). A blue/white screening test is used to confirm a status of genetic recombination.

In this embodiment of this instant disclosure, the pCRISPR::LacZ plasmid is the guide RNA (gRNA) which can identify the LacZ gene plasmid. In other words, the guide RNA is expressed from the pCRISPR::LacZ plasmid. In addition, in this embodiment of this instant disclosure, an electroporation method is used to transform the pCas9 plasmid, the pKD46 plasmid, the pCRISPR::LacZ plasmid, and the exogenous DNA into the E. coli. The theory of the electroporation method is, an electric field is used to generate a pulse electric shock to apply on a bacterium in a very short time (microseconds to milliseconds), so that the bacterium is under an environment with high-voltage and low-capacitance. When a cell membrane of the bacterium is stimulated from the electric field, the cell membrane will generate an electrical potential to cause a structure of the cell membrane to be changed. At this time, a mechanical pressing force is generated to compress and thin the cell membrane. For this reason, lipids of the cell membrane are locally cracked, proteins on the cell membrane are structurally changed, and numerous tiny holes are transitorily generated, such that exogenous macromolecules and DNA fragments can enter into the bacterium via the transitory tiny holes.

The culture medium of the first embodiment in this instant disclosure contains IPTG, X-gal, Km, and Tc. Since the IPTG is not metabolized by E. coli, it is not a variable in the experiment. The IPTG is used as an inducer of the Lac operon in the first embodiment of this experiment. The X-gal is an organic compound composed from the galactose connecting to the substituted indole. Therefore, the X-gal is a simulated lactose which is used to detect activities of a variety of bacterial enzymes. β-galactosidase is used to hydrolyze the β-glycosidic bond in a D-lactose. When the X-gal is hydrolyzed by the β-galactosidase, the galactose and the 5-bromo-4-chloro-3-hydroxyindole are generated, and the 5-bromo-4-chloro-3-hydroxyindole is spontaneously dimerized and oxidized to form the 5,5′-dibromo-4,4′-dichloro-indigo which is an insoluble dark blue product. The X-gal itself is colorless, but its product is a clear blue color. Hence, the X-gal can be used to detect the existence and the activity of β-galactosidase. For this reason, the X-gal is used in the blue/white screening test in the embodiment of this instant disclosure. In the blue/white screening test, the blue colony represents that the exogenous DNA has not been inserted into the bacterial cell, that is, the genetic recombination was not successful. The white colony represents that the exogenous DNA has been inserted into the bacterial cell, that is, the genetic recombination was successful. In the first embodiment of this instant disclosure, the Km contained in the culture medium is an aminoglycoside antibiotic, the Km inhibits the formation and synthesis of the 70S initiation complex in a ribosome, and induces the release of initial fMet-tRNA from a complex, so as to obstruct the protein synthesis to further inhibit other bacteria growth. In the first embodiment of this instant disclosure, the Tc contained in the culture medium is a polyketide antibiotic. The Tetracycline can bind to the 30S subunit in a ribosome to block the t-RNA binding to the A-site of the ribosome, so as to impede the protein synthesis to further inhibit other bacteria growth and inhibit against a variety of bacterial infections. The antibiotics such as Km and Tc are added in the culture medium in this instant disclosure to prevent the E. coli from being polluted by other bacteria. At the same time, the bacterial cells carrying the pCRISPR::LacZ plasmid (having the Km^{R} antibiotic gene) and inserted by the exogenous DNA (having the Tc^{R} antibiotic gene) are selected.

A CRISPR/Cas9 technique is used in the first embodiment of this instant disclosure to conduct the genome editing of E. coli, and the experimental steps, conditions, and results are described in detail in <Experiment 1>.

<Experiment 1>

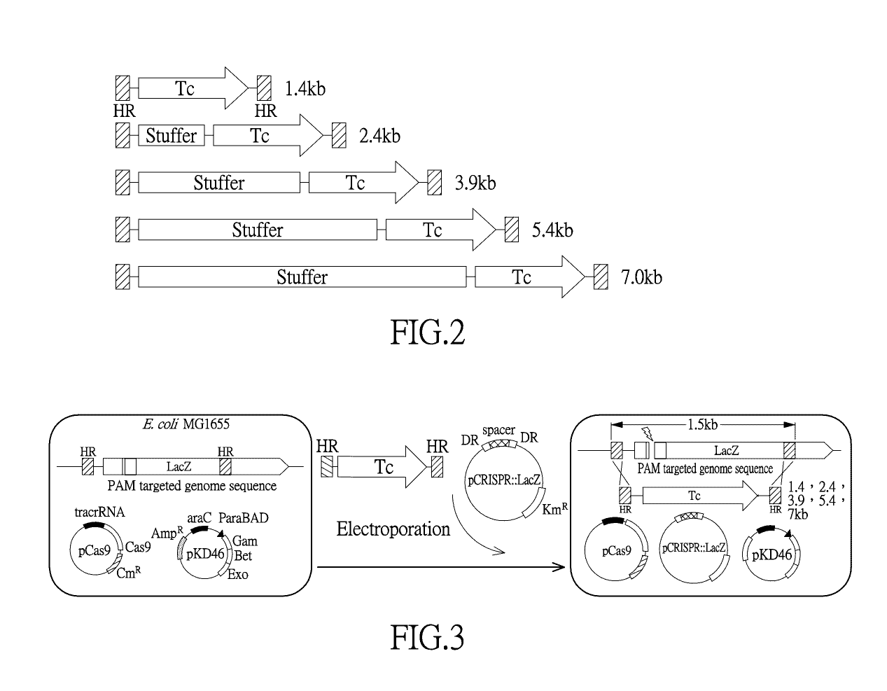

Please refer to FIGS. 2 and 3. FIG. 2 shows a schematic view of various lengths of exogenous DNA of a first embodiment in the instant disclosure, and FIG. 3 shows a schematic view of a method for bacterial genome editing of a first embodiment in the instant disclosure. The E. coli is a bacterial strain of MG1655 which is used in the bacterial genome editing in this instant disclosure. In an experimental group of this instant disclosure, the Cas9 protein expressed from the pCas9 plasmid and the pKD46 plasmid carrying a i-red system are transformed into the E. coli by the electroporation method. Next, the pCRISPR::LacZ plasmid carrying a guide RNA (gRNA) which can identify the LacZ gene and the respective exogenous DNAs (donor DNA) having lengths of 1.4, 2.4, 3.9, 5.4, and 7.0 kb are co-transformed into the E. coli carrying the pCas9 plasmid and the pKD46 plasmid by the electroporation method, and a strain broth is obtained. The strain broth is then spread on a culture medium containing IPTG; X-gal, Km, and Tc to conduct cultivation. In a control group, only the pKD46 plasmid is transformed into the E. coli, and the different lengths of exogenous DNAs (1.4, 2.4, 3.9, 5.4, and 7.0 kb) are then respectively transformed into the E. coli carrying the pKD46 plasmid by the electroporation method, so as to obtain a strain broth. The strain broth is spread on a culture medium containing IPTG, X-gal, Km, and Tc to conduct cultivation. The strain broth of the experimental group and the strain broth of the control group are cultured at 37° C. for 16 to 24 hours, and the blue/white screening test is conducted to quantitatively calculate a number of white colonies and blue colonies. The white colony represents that the genetic recombination was successful, and the blue colony represents that the genetic recombination was not successful.

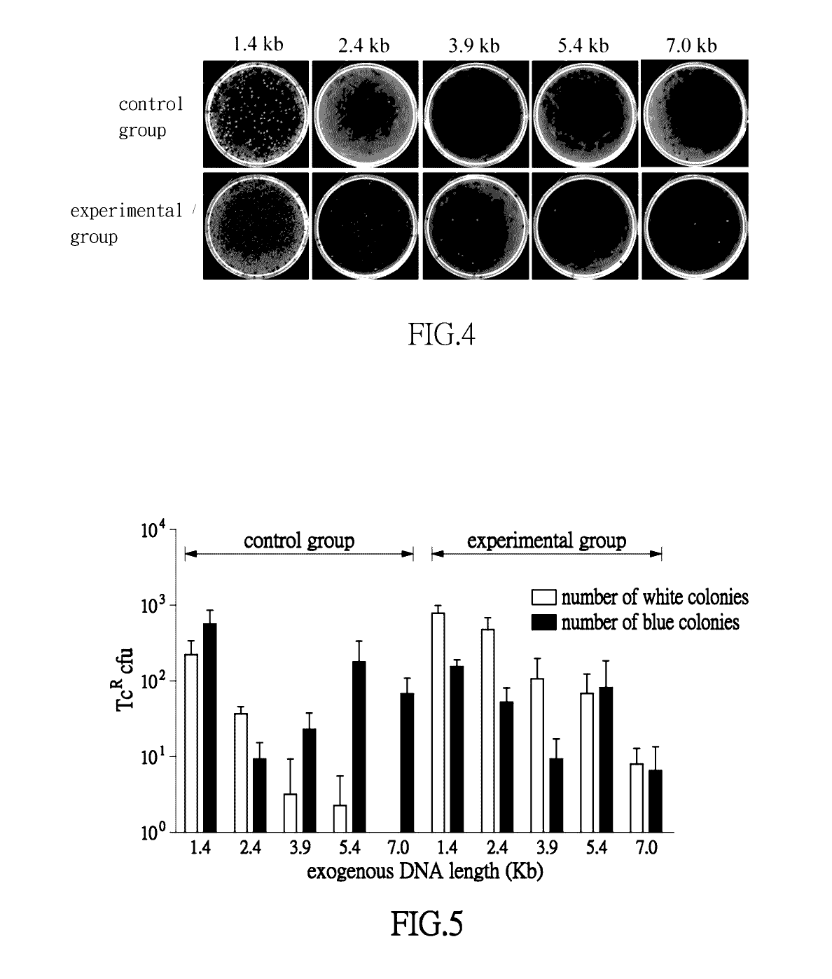

Please refer to FIG. 4, FIG. 5, and Table 1. FIG. 4 is a blue/white screening result showing the number of colonies in Experiment 1 of a first embodiment in the instant disclosure, FIG. 5 is a bar chart of blue/white screening result showing the number of colonies in Experiment 1 of a first embodiment in the instant disclosure, and Table 1 is a blue/white screening result of experimental groups and control groups.

The experimental results show that, in the control groups of 1.4, 2.4, 3.9, 5.4, and 7.0 kb, average numbers of the white colonies respectively are 223, 37, 3, 2, and 0. In the experimental groups of 1.4, 2.4, 3.9, 5.4, and 7.0 kb, average numbers of the white colonies respectively are 781, 480, 105, 68, and 8. From the experimental results, by the method for bacterial genome editing provided from the first embodiment of this instant disclosure, the average numbers of the white colonies are significantly increased (p<0.05) in all experimental groups of 1.4, 2.4, 3.9, 5.4, and 7.0 kb. According to the experimental results, a homologous recombination of the E. coli can actually be promoted by using the CRISPR/Cas9 technique to specifically cut at the specific cutting site of the LacZ gene in the E. coli, that is, a success rate of the genetic recombination can be improved. Especially, in the control groups, when the exogenous DNA has a length larger than 3.9 kb, the number of the white colonies are substantially reduced below three. This indicates that, by a conventional λ-Red homologous recombination technique, when the exogenous DNA has a length larger than 3.9 kb, the efficiency of inserting the exogenous DNA into the E. coli is decreased, so as to reduce the success rate of the genetic recombination.

Besides the number of the white colonies, the rate of picking the colonies being successfully recombined (number of the white colonies) is a key point. Even if the efficiency of genetic recombination is high, the noise (number of the blue colonies) is also high, and the recombinant colonies are difficult to be correctly picked out. In accordance with the rate of picking the colonies being successfully recombined being white colonies divided by total colonies, in the control groups of 1.4, 2.4, 3.9, 5.4, and 7.0 kb, average blue colonies respectively are 563, 9, 23, 181, and 68 (as shown in Table 1); and in the experimental groups of 1.4, 2.4, 3.9, 5.4, and 7.0 kb, average blue colonies respectively are 156, 52, 9, 82, and 7 (as shown in Table 1). Further calculating the rate of picking the successfully recombined colonies, in the control groups, the groups of 1.4 and 2.4 kb are respectively 28.3% and 80%, and the group of 3.9 kb is 12%. However, when the exogenous DNA having the length larger than 3.9 kb is in the control group, the rate of picking the successfully recombined colonies is significantly reduced to below 1% (with a large number of blue colonies). In the experimental groups, the groups of 1.4, 2.4, and 3.9 kb are 90%, and the groups of 5.4 and 7.0 kb are respectively 45 and 50%.

TABLE 1
Blue/white screening result of experimental groups and control groups.

| | Experimental Groups | | | | | Control Groups | | | | |
|---|---|---|---|---|---|---|---|---|---|---|
| | 1.4 kb | 2.4 kb | 3.9 kb | 5.4 kb | 7.0 kb | 1.4 kb | 2.4 kb | 3.9 kb | 5.4 kb | 7.0 kb |
| Number of | 781 | 480 | 105 | 68 | 8 | 223 | 37 | 3 | 2 | 0 |
| White | | | | | | | | | | |
| Colonies | | | | | | | | | | |
| Number of | 156 | 52 | 9 | 82 | 7 | 563 | 9 | 23 | 181 | 68 |
| Blue Colonies | | | | | | | | | | |

| | | | | | | | |
|---|---|---|---|---|---|---|---|
| Success Rate | about 90% | 45% | 50% | 28.3% | 80% | 12% | below 1% |
| of Genetic | | | | | | | |
| recombination | | | | | | | |

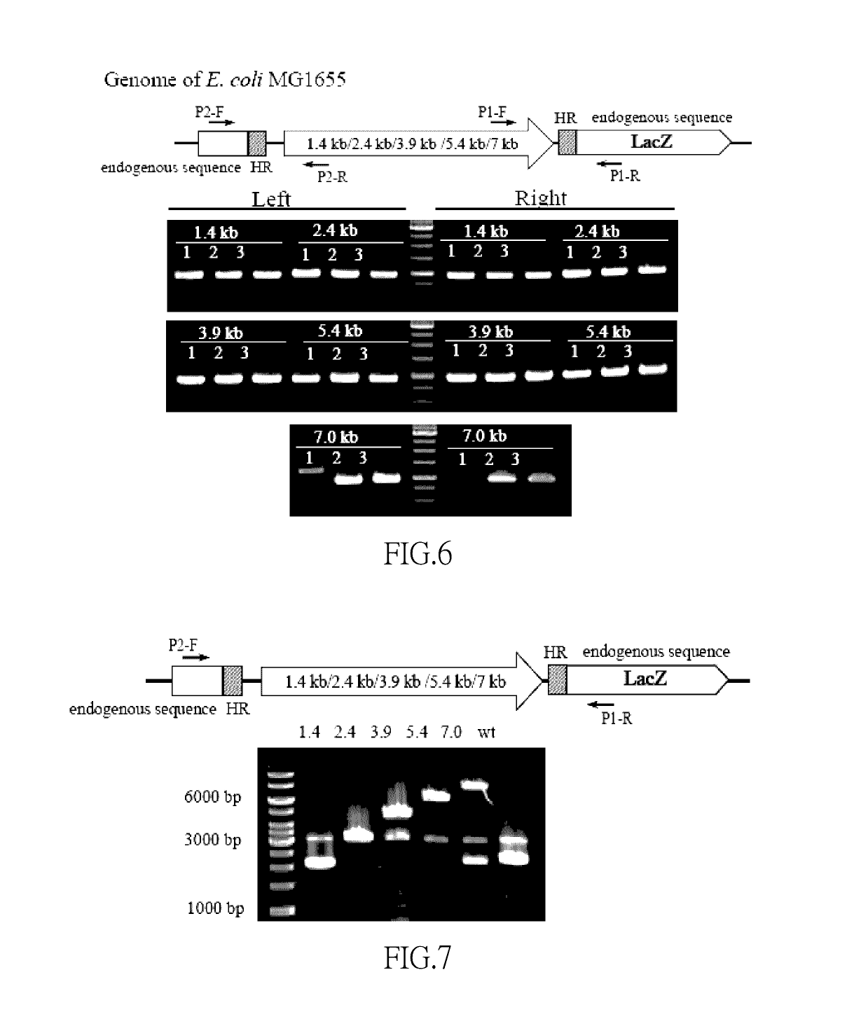

Please refer to FIGS. 6 and 7. FIG. 6 is a result of a polymerase chain reaction based colonies rapid test in Experiment 1 of a first embodiment in the instant disclosure, and FIG. 7 is an analysis result of an exogenous DNA inserting into a chromosome in Experiment 1 of a first embodiment in the instant disclosure.

In order to further confirm that the exogenous DNA has been inserted into the right location of the chromosome, two sets of primers are designed (as shown in the above FIG. 6). In the experimental group, 3 to 5 white colonies are randomly picked, the exogenous DNAs are inserted into left and right seams of the chromosome, and a colony PCR is conducted (polymerase chain reaction for colonies rapid test). If the exogenous DNA has been inserted into the right location, a PCR signal of 1 kb will be generated. In the Colony PCR, the bacterial cell is lysed by heating, and the DNA of the bacterial cell is exposed which serves as a template to conduct a PCR amplification. By this way, a genome DNA does not have to be extracted and does not have to be determined by a restriction enzyme. The screening of genetic recombination or DNA sequence analysis is usually conducted by the Colony PCR. Results can be obtained by the Colony PCR analysis (below FIG. 6), and in the groups of 1.4, 2.4, and 3.9 kb, all of the colonies express correct PCR signals. In the group of 5.4 kb, there are four colonies expressing the correct PCR signals in the five colonies, and a correct rate is 80%. In the group of 7.0 kb, there are three colonies expressing the correct PCR signals in the five colonies, and a correct rate is 60%. Finally, the primers being complementary to outer seams of the chromosome are used (above in FIG. 7) to insert whole PCR fragments into the exogenous DNA of the chromosome, so as to confirm the exogenous DNA has been completely inserted into the chromosome (below in FIG. 7). The above results can confirm that, in the experimental groups, all of the exogenous DNA having different sizes can be correctly inserted into the Lac Z site on the chromosome in the E. coli.

In the method for bacterial genome editing provided from this instant disclosure, the CRISPR/Cas9 technique is used to conduct the bacterial genome editing, in which, the pCas9 plasmid, the pKD46 plasmid, and the pCRISPR::LacZ plasmid are co-transformed into the E. coli. In addition, the pCas9 plasmid, the pKD46 plasmid, and the pCRISPR::LacZ plasmid cooperate with each other, and coordinate the cultivation conditions of the culture medium containing IPTG, X-gal, Km, and Tc, such that a DNA having large fragment (e.g., 7 kb) can be successfully inserted into the E. coli. At the same time, the success rate of the genetic recombination can be increased.

### Second Embodiment

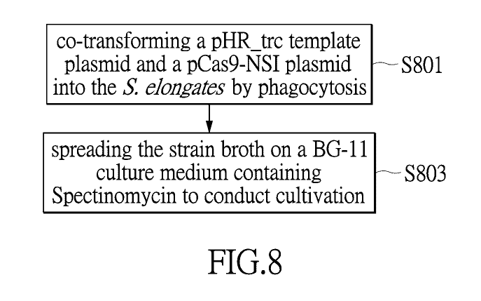

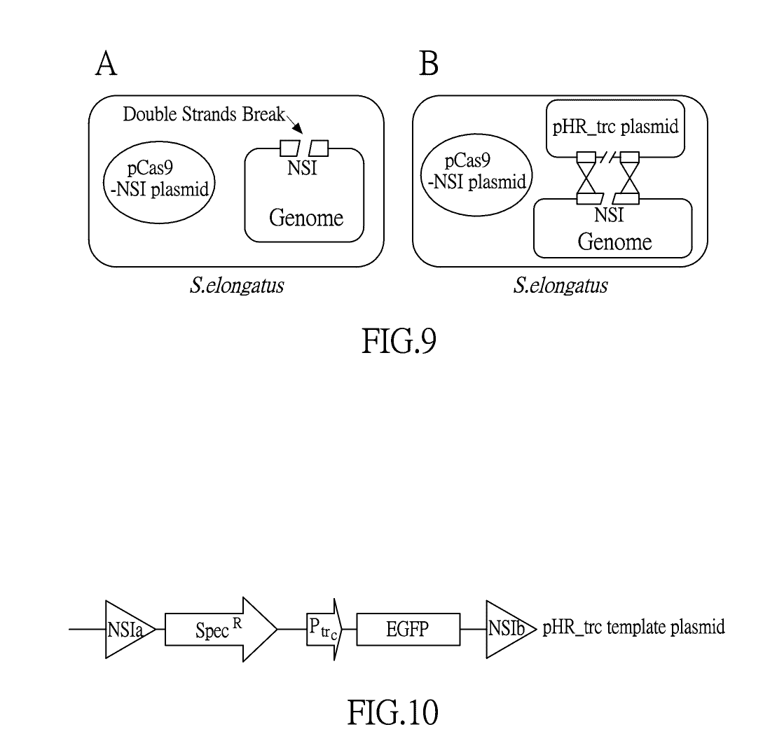

Please refer to FIGS. 8 and 9. FIG. 8 shows a flow chart of a method for bacterial genome editing of a second embodiment in the instant disclosure, and S801 and S803 represent steps of the flow chart. FIG. 9 shows a schematic view of a method for bacterial genome editing of a second embodiment in the instant disclosure.

A second embodiment in the instant disclosure provides a method for bacterial genome editing. The bacterial cell is a cyanobacteria in the second embodiment, and the cyanobacteria is a bacterial strain of S. elongates PCC7942 (herein referred to as “S. elongates”).

The method for bacterial genome editing of the second embodiment in the instant disclosure includes the following steps: providing a S. elongates; co-transforming a pHR_trc template plasmid and a pCas9-NSI plasmid into the S. elongates by a phagocytosis method to correctly bind to a predetermined position to further conduct a genetic recombination process, wherein the pHR_trc template plasmid is used as a homologous recombination template, the pCas9-NSI plasmid is used for correctly binding to the predetermined position, and the predetermined position is a double-strand break locus; obtaining a strain broth; and spreading the strain broth on a culture medium to conduct cultivation at 30° C. under light intensity of 50 μmol m-2 s, wherein the culture medium is a BG-11 culture medium containing Spectinomycin.

The CRISPR/Cas9 technique is used in the second embodiment of this instant disclosure to conduct the genome editing of S. elongates, and the experimental steps, conditions, and results are described in detail in <Experiment 2>.

<Experiment 2>

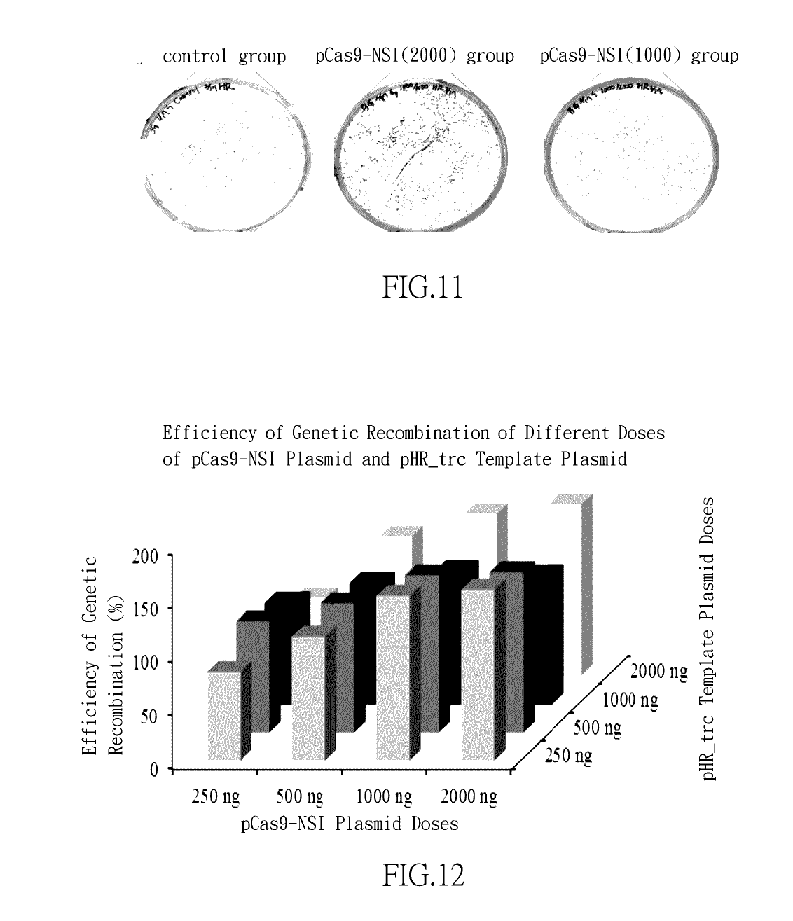

Please refer to FIGS. 10 to 12 and Table 2. FIG. 10 shows a schematic view of a template plasmid in Experiment 2 of a second embodiment in the instant disclosure, FIG. 11 is a result of homologous recombination efficiency of a cyanobacteria in Experiment 2 of a second embodiment in the instant disclosure, FIG. 12 is a bar chart showing the homologous recombination efficiency in Experiment 2 of a second embodiment in the instant disclosure, and Table 2 is a composition table of the BG-11 culture medium.

In Experiment 2, the cyanobacteria is a bacterial strain of S. elongatus PCC7942.

The S. elongatus is a bacterial strain of PCC7942 which is used in the bacterial genome editing in the second embodiment of this instant disclosure by the CRISPR/Cas9 technique, and the S. elongates is used to test the homologous recombination, that is, to test the efficiency of genetic recombination. As shown in FIG. 10, the pHR_trc template plasmid is used as the homologous recombination template, and contains a Spectinomycin resistance gene Spec^{R}, a fluorescent protein EGFP, and a homologous recombination region NSIa and NSIb.

Firstly, the pHR_trc template plasmid is used as the homologous recombination template, and a control group references to a conventional method so that only a pHR_trc template plasmid of 1000 ng is inserted into the S. elongates. Experimental groups contain a pCas9-NSI (1000) group and a pCas9-NSI (2000) group. The pCas9-NSI (1000) group contains a pHR_trc template plasmid of 1000 ng and a pCas9-NSI plasmid of 1000 ng. The pCas9-NSI (2000) group contains a pHR_trc template plasmid of 1000 ng and a pCas9-NSI plasmid of 2000 ng. In the experiment, a method of co-transforming the pHR_trc template plasmid and the pCas9-NSI plasmid of the CRISPR/Cas9 system into the S. elongates is phagocytosis. When a strain broth is obtained, the strain broth is spread on the BG-11 culture medium containing Spectinomycin, and colony growth results are observed. BG-11 culture medium is most widely used for culturing the S. elongates, and its composition is shown in Table 2. After screening by an antibiotic (e.g., Spectinomycin), the last surviving number of the S. elongates colonies represent the number of the S. elongates that have had the homologous recombination successfully conducted so that the exogenous DNA is inserted into a genome of the S. elongates. That is, the number of the S. elongates that have had the genetic recombination successfully conducted.

TABLE 2
Compositions of the BG-11 culture medium.

| | Concentrations |
|---|---|
| Compositions | (g/L) |
| H₃BO₃ | 2.86 × 10⁻³ |
| MnCl₂•4H₂O | 1.81 × 10⁻³ |
| ZnSO4•7H₂O | 0.22 × 10⁻³ |
| NaMoO₄•2H₂O | 0.39 × 10⁻³ |
| CuSO₄•5H₂O | 7.9 × 10⁻⁵ |
| Co(NO₃)₂•6H₂O | 4.9 × 10⁻⁵ |
| MgSO₄ | 0.0361 |
| CaCl₂•2H₂O | 0.0272 |
| NaNO₃ | 1.5 |
| Na₃C₆H₅O₇•2H₂O | 2.646 × 10⁻³ |
| diNaEDTA | 0.001 |
| K₂HPO₄ | 0.040 |
| Na₂CO₃ | 0.020 |
| Ferric ammonium citrate | 0.012 |

As shown in FIG. 11, the experimental results indicated that, compared to the number of colonies in the experimental group and the number of colonies in the control group, the experimental group has more number of colonies. That is, in the experimental group, the S. elongates was not affected by the antibiotic, and have more surviving numbers after genetic recombination. The highest number of colonies of the experimental group is up to 155% of the control group.

Next, a dose of the best optimal condition is found out by adjusting different doses of the pCas9-NSI plasmid and the pHR_trc template plasmid. Therefore, groups of 250 ng, 500 ng, 1000 ng, and 2000 ng of the pCas9-NSI plasmid and groups of 250 ng, 500 ng, 1000 ng, and 2000 ng of the pHR_trc template plasmid are designed to observe the efficiencies of the genetic recombination after interactions. As shown in FIG. 12, the experimental results indicate that, the group of 250 ng of the pCas9-NSI plasmid is hardly promoting the genetic recombination. However, when the dose of the pCas9-NSI plasmid is increased to 500 ng, the efficiency of the genetic recombination is also gradually increased, and the highest efficiency value is up to 130±20%. But there is no significant difference between the groups of 1000 ng and 2000 ng of the pCas9-NSI plasmid. This means, when the dose of the pCas9-NSI plasmid is up to 1000 ng, the effectiveness the pCas9-NSI plasmid is gradually reaching saturation. Additionally, in the groups of the pHR_trc template plasmid, there is no significant difference, which means, via the method for bacterial genome editing provided from the second embodiment of this instant disclosure, the efficiency of the genetic recombination can be increased just by using a lesser amount of the pHR_trc template plasmid.

Thus, via the method for bacterial genome editing provided from the second embodiment of this instant disclosure, the exogenous DNA can be promoted to be inserted into the genome of the S. elongates and conduct the homologous recombination, so as to increase the efficiency of genetic recombination of the S. elongates. Furthermore, a material cost can be saved by just using a lesser amount of the pHR_trc template plasmid to reach the effect of genetic recombination.

### Possible Efficacies of the Embodiments

In summary, the beneficial effects of the instant disclosure are, by the method for bacterial genome editing of the embodiment in this instant disclosure, the large DNA fragments can be inserted into the bacterium, and genetic recombination in the bacterium can be successfully conducted. Therefore, the method for bacterial genome editing of the embodiment in this instant disclosure can be used for increasing the convenience of genome editing in the bacterium, and can speed up the genome editing in the bacterium, so as to modify a bacterial DNA. In the future, the method for bacterial genome editing of the embodiment in this instant disclosure can be used for regulating a bacterial metabolic pathway to achieve a purpose of producing bio-chemicals, so as to replace conventional oil cracking which entails a heavy pollution process.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. A method for bacterial genome editing, comprising following steps:
providing a bacterial cell;
transforming a pCas9 plasmid and a pKD46 plasmid into the bacterial cell;
co-transforming a pCRISPR::LacZ plasmid and an exogenous DNA into the bacterial cell carrying the pCas9 plasmid and the pKD46 plasmid, so as to obtain a strain broth; and
spreading the strain broth on a culture medium to conduct cultivation.

2. The method for bacterial genome editing as claimed in claim 1, wherein after the steps of co-transforming the pCRISPR::LacZ plasmid and the exogenous DNA into the bacterial cell carrying the pCas9 plasmid and the pKD46 plasmid, further comprises following steps:
using a Cas9 protein and a guide RNA which can identify a LacZ gene to specifically cut at a specific cutting site of the LacZ gene in the bacterial cell; and
inserting the exogenous DNA into the specific cutting site of the LacZ gene in the bacterial cell, and obtaining the strain broth.

3. The method for bacterial genome editing as claimed in claim 1, wherein the bacterial cell is an Escherichia coli.

4. The method for bacterial genome editing as claimed in claim 3, wherein the Escherichia coli is a bacterial strain of MG1655.

5. The method for bacterial genome editing as claimed in claim 1, wherein the guide RNA is expressed from the pCRISPR::LacZ plasmid which can identify the LacZ gene.

6. The method for bacterial genome editing as claimed in claim 1, wherein a method of transforming the pCas9 plasmid and the pKD46 plasmid into the bacterial cell is an electroporation method.

7. The method for bacterial genome editing as claimed in claim 1, wherein a method of co-transforming the pCRISPR::LacZ plasmid and the exogenous DNA into the bacterial cell carrying the pCas9 plasmid and the pKD46 plasmid is an electroporation method.

8. The method for bacterial genome editing as claimed in claim 1, wherein the culture medium contains isopropyl β-D-1-thiogalactopyranoside, 5-bromo-4-chloro-3-indolyl-β-D-galactopyranoside, Kanamycin, and Tetracycline.

9. The method for bacterial genome editing as claimed in claim 1, wherein a cultivation condition of the strain broth is at 37° C. for 16 to 24 hours.

10. A method for bacterial genome editing, comprising following steps:
providing a bacterial cell;
co-transforming a pHR_trc template plasmid and a pCas9-NSI plasmid into the bacterial cell to obtain a strain broth; and
spreading the strain broth on a culture medium to conduct cultivation.

11. The method for bacterial genome editing as claimed in claim 10, wherein the bacterial cell is a cyanobacteria.

12. The method for bacterial genome editing as claimed in claim 11, wherein the cyanobacteria is a bacterial strain of S. elongatus PCC7942.

13. The method for bacterial genome editing as claimed in claim 10, wherein the pHR_trc template plasmid is used as a homologous recombination template.

14. The method for bacterial genome editing as claimed in claim 10, wherein the pHR_trc template plasmid contains a Spectinomycin resistance gene Spec^{R}, a fluorescent protein EGFP, and a homologous recombination region NSIa and NSIb.

15. The method for bacterial genome editing as claimed in claim 10, wherein the pCas9-NSI plasmid is used for correctly binding to a predetermined position to conduct a genetic recombination process.

16. The method for bacterial genome editing as claimed in claim 15, wherein the predetermined position is a double-strand break locus.

17. The method for bacterial genome editing as claimed in claim 10, wherein a method of co-transforming the pHR_trc template plasmid and the pCas9-NSI plasmid into the bacterial cell is phagocytosis.

18. The method for bacterial genome editing as claimed in claim 10, wherein the culture medium is a BG-11 culture medium containing Spectinomycin.
